(19) United States
(12) Patent Application Publication
(10) Pub. No.: US 2011/0301316 A1
(43) Pub. Date: Dec. 8, 2011

(54) METHOD FOR MANUFACTURING BIOMASS-DERIVED METHYL METHACRYLATE

(75) Inventor: Jean-Luc Dubois, Millery (FR)

(73) Assignee: Arkema France, Colombes (FR)

(21) Appl. No.: 13/143,448

(22) PCT Filed: Jan. 5, 2010
(86) PCT No.: PCT/FR2010/050003

§ 371 Date: Aug. 29, 2011

(30) Foreign Application Priority Data
Jan. 6, 2009 (FR) 0950028

(51) Int. Cl. C08F 120/10; C07C 67/42

(52) U.S. Cl. 526/329.7; 560/208

(57) ABSTRACT

Process for the manufacture of methyl methacrylate by oxidation of methacrolein to methacrylic acid and esterification of the latter with methanol, characterized in that at least one fraction of at least one from among methacrolein and methanol in this reaction was obtained by a reaction or a sequence of reactions starting from biomass.

The present invention relates to a process for the manufacture of a biomass-derived methyl methacrylate.

Methyl methacrylate is the raw material of numerous polymerization or copolymerization reactions.

It is the monomer for the manufacture of poly(methyl methacrylate) (PMMA), known under the Altuglas® and Plexiglas® trade names. It is provided in the form of powders, granules or sheets, the powders or granules being used for the molding of various items, such as items for the motor vehicle industry, household items and office items, and the sheets finding use in signs and displays, in the fields of transport, building, lights and sanitary ware, as anti-noise walls, for works of art, flat screens, etc.

Methyl methacrylate is also the raw material for the organic synthesis of higher methacrylates which, like it, are used in the preparation of acrylic emulsions and acrylic resins, act as additives for poly(vinyl chloride), are used as comonomers in the manufacture of numerous copolymers, such as methyl methacrylate/butadiene/styrene copolymers, act as additives for lubricants and have many other applications, among which may be mentioned medical prostheses, flocculants, cleaning products, etc. Acrylic emulsions and resins have applications in the fields of paints, adhesives, paper, textiles, inks, etc. Acrylic resins are also used in the manufacture of sheets having the same applications as PMMA.

Methyl methacrylate can be obtained in various ways, one of these consisting of an oxidation of methacrolein to methacrylic acid and esterification of the latter with methanol.

Patent Applications EP 1 994 978 and EP 1 995 231 describe a process for manufacturing methyl methacrylate by esterification of methacrylic acid with methanol, the methacrylic acid being obtained by oxidation of methacrolein originating from the oxidation of isobutene.

In documents EP 1 813 586 and U.S. Pat. No. 3,819,685, the oxidation of methacrolein is carried out in the presence of methanol resulting directly in the production of methyl methacrylate.

In document GB 2 094 782, methyl methacrylate is produced from methacrolein derived from isobutyraldehyde obtained by hydroformylation of propylene in the presence of hydrogen and carbon monoxide.

In document EP 058 927, methacrolein is obtained by reaction of propanal with formol and a secondary amine in the presence of an acid.

The raw materials used for these syntheses of methyl methacrylate are mainly of petroleum origin or of synthetic origin, thus comprising numerous sources of emission of CO₂, which consequently contribute to increasing the greenhouse effect. Given the decrease in world oil reserves, the source of these raw materials will gradually become exhausted.

The raw materials resulting from biomass are a renewable source and have a reduced impact on the environment. They do not require all the stages of refining, which are very expensive in terms of energy, of oil products. The production of fossil CO₂ is reduced, so that they contribute less to climate warming. The plant, in particular for the growth thereof, has consumed atmospheric CO₂ at a rate of 44 g of CO₂ per mole of carbon (or per 12 g of carbon). Thus, the use of a renewable source begins by reducing the amount of atmospheric CO₂. Plant materials exhibit the advantage of being able to be cultivated in large amounts, according to demand, over most of the world.

It thus appears necessary to have available processes for the synthesis of methyl methacrylate which are not dependent on raw materials of fossil origin but which instead use biomass as raw material.

The term “biomass” is understood to me-an raw material, of plant or animal origin, produced naturally. This plant material is characterized in that the plant, for the growth thereof, has consumed atmospheric CO₂ while producing oxygen. Animals, for their growth, have for their part consumed this plant raw material and have thus taken in the carbon derived from atmospheric CO₂.

The aim of the present invention is thus to respond to certain concerns for sustainable development.

A subject matter of the present invention is thus a process for the manufacture of methyl methacrylate by oxidation of methacrolein to methacrylic acid and esterification of the latter with methanol, characterized in that at least one fraction of at least one from among methacrolein and methanol in this reaction was obtained by a reaction or a sequence of reactions starting from biomass.

Said oxidation and said esterification can be carried out in two successive steps or else simultaneously.

At least one fraction of methanol was able to be obtained by pyrolysis of wood or by gasification of any material of animal and/or plant origin resulting in a syngas essentially composed of carbon monoxide and hydrogen, or by fermentation starting from plant crops, such as wheat, sugar cane or beet, giving fermentable products and thus alcohol, it also being possible for at least one fraction of syngas for preparing the methanol to originate from the recovery of spent liquor and bleaching liquor from the manufacture of cellulose pulps.

In accordance with a first embodiment, at least one fraction of methacrolein was able to be obtained by oxidation of at least one from among isobutene, tert-butanol and/or a mixture of the two, the isobutene, where appropriate as a mixture with tent-butanol, possibly resulting from the dehydration of isobutanol, it being possible for at least one fraction of isobutanol to have been obtained by distillation of a fusel oil, and/or by fermentation, in the presence of at least one yeast, of at least one plant material, which is generally in a hydrolyzed form before the fermentation, the fermentation being followed by a distillation step in order to recover the isobutanol in the form of an aqueous solution, which solution is subsequently subjected to a concentration step, and/or by condensation of methanol with ethanol, the methanol and/or the ethanol being derived from biomass.

In accordance with a second embodiment of the invention, at least one fraction of methacrolein was able to be obtained by oxidizing dehydrogenation of isobutyraldebyde, at least one fraction of this possibly resulting from the reaction of propylene with a syngas and/or from the oxidation of isobutanol,

it being possible for at least one fraction of isobutanol to have been obtained by distillation of a fusel oil, and/or by fermentation, in the presence of at least one yeast, of at least one plant material, which is generally in a hydrolyzed form before the fermentation, the fermentation being followed by a distillation step in order to recover the isobutanol in the form of an aqueous solution, which solution is subsequently subjected to a concentration step, and/or by condensation of methanol with ethanol, the methanol and/or the ethanol being derived from biomass;
it being possible for at least one fraction of syngas to originate from the gasification of any material of animal or plant origin and/or from the recovery of spent liquor and bleaching liquor from the manufacture of cellulose pulps.

At least one fraction of propylene was able to be obtained by dehydration of isopropanol, itself obtained by biomass fermentation, or by metathesis reaction of ethylene and but-2-ene, themselves obtained by dehydration of a mixture of alcohols, comprising at least ethanol and butan-1-ol, resulting from biomass fermentation.

In accordance with a third embodiment, at least one fraction of methacrolein was able to be obtained by reaction of propanaldehyde over formaldehyde,

at least one fraction of propanaldehyde possibly resulting from the hydrogenation of acrolein, at least one fraction of the latter originating from the dehydration of glycerol, at least one fraction of the latter having been able to be obtained as a by-product of the manufacture of biofuels starting from oleaginous plants, such as rape, sunflower or soya, comprising triglycerides, a hydrolysis or a transesterification of these triglycerides making it possible to form glycerol in addition to fatty acids and fatty esters respectively; and
at least one fraction of formaldehyde by oxidation of methanol, at least one fraction of the methanol used having been obtained by pyrolysis of wood or by gasification of any material of animal or plant origin resulting in a syngas essentially composed of carbon monoxide and hydrogen, or by fermentation starting from plant crops, such as wheat, corn, sugar cane or beet, giving fermentable products and thus alcohol.

Another subject matter of the present invention is the use of the methyl methacrylate manufactured by the process as defined above as monomer for the manufacture of poly(methyl methacrylate), as raw material for the organic synthesis of higher methacrylates, as product used in the preparation of acrylic emulsions and acrylic resins, as additive for poly(vinyl chloride), as comonomer in the manufacture of copolymers, and as additive for lubricants.

### Upgrading of Biomass to Methanol

As indicated above, the methanol is obtained by pyrolysis of wood or by gasification of any material of animal or plant origin, resulting in a syngas essentially composed of carbon monoxide and hydrogen, which is optionally reacted with water by the water gas shift reaction in order to adjust the H₂/CO ratio to within the proportions appropriate to the synthesis of the methanol, or by fermentation starting from plant crops, such as wheat, corn, sugar cane or beet, giving fermentable products and thus alcohol.

The materials of animal origin are, as nonlimiting examples, fish oils and fats, such as cod liver oil, whale oil, sperm whale oil, dolphin oil, seal oil, sardine oil, herring oil or shark oil, oils and fats of bovines, porcines, caprines, equids, and poultry, such as tallow, lard, milk fat, pig fat, chicken, cow, pig or horse fats, and others.

The materials of plant origin are, as nonlimiting examples, lignocellulose residues from agriculture, cereal straw fodder, such as wheat straw fodder or corn straw or ear residue fodder; cereal residues, such as corn residues; cereal flours, such as wheat flour; cereals, such as wheat, barley, sorghum or corn; wood, or wood waste and scraps; grains; sugar cane or sugar cane residues; pea tendrils and stems; beets or molasses, such as beet molasses; Jerusalem artichokes; potatoes, potato haulms or potato residues; starch, mixtures of cellulose, hemicellulose and lignin; and black liquor from the paper-making industry, which is a material rich in carbon.

According to a specific embodiment of the invention, the syngas for preparing the methanol originates from the recovery of spent liquor and bleaching liquor from the manufacture of cellulose pulps. Reference may be made to the documents EP 666 831 and U.S. Pat. No. 7,294,225 of Chemrec, which describe, in particular, the gasification of spent liquors from the manufacture and bleaching of cellulose and the production of methanol, and to pages 92-105 of the work Procédés de pétrochimie—Caractéristiques techniques et économiques—Tome I—Editions Technip—le gaz de synthèse et ses dérivés [Petrochemical processes—Technical and Economic Characteristics—Volume 1—Published by Technip—Syngas and its derivatives], which relates to the production of methanol from syngas.

### Upgrading of Biomass to Isobutanol by Distillation of Fusel Oils Also Known as Fusel Alcohols.

Ethanolic fermentation, fermentation of biomass such that the sugar gives ethanol, results in alcohols that are heavier than ethanol, in a proportion of the order of 5 kg per tonne of ethanol. This mixture of alcohols mainly consists of alcohols having 5, 4 and 3 carbon atoms such as amyl and isoamyl alcohols, isobutanol and propanol. It is then possible to isolate the isobutanol from this mixture of alcohols, especially via distillation technologies.

### Upgrading of Biomass to Isobutanol by Condensation of Methanol With Ethanol

The condensation of methanol with ethanol results, according to Guerbet reactions, in a mixture of propanol and isobutanol (2-methylpropan-1-ol) with the minority presence of other branched alcohols such as 2-methylbutan-1-ol. The composition of this mixture of alcohols depends, on the one hand, on the nature of the catalyst(s) used for the Guerbet reactions and, on the other hand, on the ratio between the two reactants, methanol and ethanol. It is then possible to isolate the isobutanol from this mixture of alcohols, for example via distillation technologies. The reaction mechanism of the Guerbet reactions passes through the formation of formaldehyde and acetaldehyde from methanol and ethanol respectively, which condense to produce propenal, which is reduced to propanol. The condensation of formaldehyde with propanal results in isobutanol.

These various reactions and the implementation conditions thereof are described in particular in the article by E. S. Olson et al., Applied Biochemistry and Biotechnology, Vol. 113-116, 2004, p. 913-930.

For the Guerbet reaction, it was possible to obtain methanol from biomass as described above, and ethanol by fermentation of plant material that can be chosen in particular from sugars, starch and the plant extracts comprising them, among which may be mentioned beet, sugar cane, cereals, such as wheat, barley, sorghum or corn, and potatoes without this list being limiting. It can alternatively be biomass (mixture of cellulose, hemicellulose and lignin). Ethanol is then obtained by fermentation, for example, using Saccharomyces cerevisiae or its mutant.

These fermentation methods are well known to a person skilled in the art. They include, for example the fermentation of plant materials in the presence of one or more yeasts or mutants of these yeasts (microorganisms naturally modified in response to a chemical or physical stress), followed by distillation in order to recover the alcohol, in particular ethanol, in the form of a more concentrated aqueous solution, which solution is subsequently treated for the purpose of further increasing its molar concentration of alcohol such as ethanol. The ethanol is generally obtained as a mixture with heavier alcohols, known as fusel alcohols, the composition of which depends on the plant material used and on the fermentation process. It is possible to purify the ethanol produced by fermentation, for example, by absorption on filters of the molecular sieve, carbon black or zeolite type.

### Upgrading of Biomass to Propylene

As indicated above, according to a first variant, propylene is obtained by dehydration of isopropanol, the isopropanol being obtained by fermentation of renewable raw materials in the presence of one or more appropriate microorganisms, this microorganism may optionally have been modified naturally by a chemical or physical stress, or genetically, it then being referred to as a mutant.

As biomass, it will be possible to use plant materials; materials of animal origin or materials of plant or animal origin resulting from reclaimed materials (recycled materials).

Within the meaning of the invention, the materials of plant origin contain at least sugars and/or polysaccharides such as starch, cellulose or hemicellulose.

The plant materials containing sugars are essentially sugar cane and sugar beet, mention may also be made of maple, date palm, sugar palm, sorghum or American agave; the plant materials containing starches are essentially cereals and legumes, such as corn, wheat, barley, sorghum, soft wheat, rice, potato, cassava or sweet potato, or else algae.

Use may also be made, as renewable raw materials, of cellulose or hemicellulose, which can be converted to sugar-comprising materials in the presence of appropriate microorganisms. These renewable materials include straw, wood or paper, which may advantageously originate from reclaimed materials.

Mention may in particular be made, among materials resulting from reclaimed materials, of plant or organic waste comprising sugars and/or polysaccharides.

Preferably, the renewable raw materials are plant materials.

In the case of polysaccharides, the plant material used is generally in hydrolyzed form before the fermentation step. This preliminary hydrolysis step thus enables, for example, the saccharification of starch in order to convert it to glucose, or the conversion of sucrose to glucose.

Advantageously, the microorganisms used for the fermentation are Clostridium beijerinckii, Clostridium aurantibutyricum or Clostridium butylicum and also the mutants thereof, preferably immobilized on a support of the polymer fiber or calcium type.

The fermentation of these raw materials essentially results in the production of isopropanol and/or butanols, optionally with acetone.

The fermentation step is advantageously followed by a purification step, for example a distillation intended to separate the isopropanol from the other alcohols.

The dehydration is carried out in the presence of oxygen and water using a catalyst based on γ-alumina, such as the catalyst sold by Eurosupport under the trade name ESM 110® (undoped trilobe alumina containing little—around 0.04%—residual Na₂O).

The operating conditions for the dehydration form part of the general knowledge of a person skilled in the art; by way of indication, the dehydration is generally carried out at a temperature of around 400° C.

According to a second variant, propylene is obtained by metathesis reaction of ethylene and but-2-ene, themselves being obtained by dehydration of a mixture of alcohols, comprising at least ethanol and butan-1-ol, resulting from the fermentation of biomass using Clostridium beijerinckii or a mutant thereof.

The dehydration of ethanol and butan-1-ol with a view to producing ethylene and but-1-ene is carried out under the same conditions as the dehydration of isopropanol described above. Next, a hydroisomerization reaction of but-1-ene to give but-2-ene is carried out. Finally, the metathesis of ethylene and but-2-ene results in the formation of propylene.

The details of the hydroisomerization and metathesis reactions are, for example, mentioned in patent application FR 2 880 018.

### Upgrading of Biomass to Glycerol

The glycerol is obtained from oleaginous plants, such as rape, sunflower or soya, comprising oils (triglycerides) or from animal fats.

A stage of hydrolysis or transesterification of the triglycerides is carried out in order to form, with the glycerol, fatty acids and fatty esters respectively.

For example, this transesterification can be carried out by reacting the crude oil in a stirred reactor in the presence of an excess of alcohol (for example methanol), preferably with a basic catalyst (such as sodium methoxide or sodium hydroxide). In order to carry out the hydrolysis reaction, the crude oil is reacted in the presence of an excess of water, preferably with an acid catalyst. This transesterification or hydrolysis reaction is preferably carried out at a temperature of between 30 and 250° C., and preferably of between 40 and 120° C. Preferably, the reactor is fed continuously in order to keep the water/acid or alcohol/ester molar ratio greater than or equal to 2/1. At the end of the reaction, the glycerol is separated by settling from the mixture obtained.

The present invention thus makes it possible to obtain a methyl methacrylate having at least a portion of its carbons of renewable origin.

A renewable raw material is an animal or plant natural resource, the stock of which can be reconstituted over a short period on the human scale. In particular, it is necessary for this stock to be able to be renewed as quickly as it is consumed.

Unlike the materials resulting from fossil materials, renewable raw materials comprise ¹⁴C in the same proportions as atmospheric CO₂. All the samples of carbon drawn from living organisms (animals or plants) are in fact a mixture of 3 isotopes: ¹²C (representing approximately 98.892%), ¹³C (approximately 1.108%) and ¹⁴C (traces: 1.2×10⁻¹⁰%). The ¹⁴C/¹²C ratio of living tissues is identical to that of the atmosphere. In the environment, ¹⁴C exists in two predominant forms: in the inorganic form, that is to say in the form of carbon dioxide gas (CO₂), and in the organic form, that is to say in the form of carbon incorporated in organic molecules.

In a living organism, the ¹⁴C/¹²C ratio is kept constant by the metabolism as the carbon is continually exchanged with the environment. As the proportion of ¹⁴C is constant in the atmosphere, it is the same in the organism, as long as it is living, since it absorbs this ¹⁴C as it absorbs the ¹²C. The mean ¹⁴C/¹²C ratio is equal to 1.2×10⁻¹². Carbon-14 results from the bombardment of atmospheric nitrogen (14) and is spontaneously oxidized with the oxygen of the air to give CO₂. In our human history, the ¹⁴CO₂ content increased as a result of atmospheric nuclear explosions but then has not stopped decreasing after the cessation of these tests.

¹²C is stable, that is to say that the number of ¹²C atoms in a given sample is constant over time. ¹⁴C is for its part radioactive (each gram of carbon of a living being contains enough ¹⁴C isotopes to give 13.6 disintegrations per minute) and the number of such atoms in a sample decreases over time (t) according to the law:

n=no exp(−at),

in which:
no is the ¹⁴C number at the start (on the death of the creature, animal or plant),
n is the number of ¹⁴C atoms remaining after time t,
a is the disintegration constant (or radioactive constant); it is related to the half-life.

The half-life (or period) is the period of time, at the end of which any number of radioactive nuclei or unstable particles of a given entity is reduced by half by disintegration; the half-life T_{1/2} is related to the disintegration constant a by the formula aT_{1/2}=In 2. The half-life of ¹⁴C has a value of 5730 years. In 50 000 years, the ¹⁴C content is less than 0.2% of the starting content and thus becomes difficult to detect. Petroleum products or natural gas or else coal thus do not comprise ¹⁴C.

In view of the half-life (T_{1/2}) of ¹⁴C, the ¹⁴C content is substantially constant from the extraction of the renewable raw materials up to the manufacture of the methyl methacrylate according to the invention and even up to the end of its use.

The methyl methacrylate obtained according to the invention comprises organic carbon resulting from renewable raw materials; it is for this reason characterized in that it comprises ¹⁴C.

In particular, at least 1% by weight of the carbons of said methyl methacrylate is of renewable origin. Preferably, at least 20% of the carbons of said methyl methacrylate are of renewable origin. More preferably still, at least 40% of the carbons of said methyl methacrylate are of renewable origin. More particularly, at least 60% and even more specifically still at least 80% of the carbons of said methyl methacrylate are of renewable origin.

The methyl methacrylate obtained according to the invention comprises at least 0.01×10⁻¹⁰% by weight, preferably at least 0.2×10⁻¹⁰%, of ¹⁴C with regard to the total weight of carbon. More preferably still, said methyl methacrylate comprises at least 0.4×10⁻¹⁰% of ¹⁴C, more particularly at least 0.7×10⁻¹⁰% of ¹⁴C and more specifically still at least 0.9×10⁻¹⁰% of ¹⁴C.

In a preferred embodiment of the invention, the methyl methacrylate obtained according to the invention comprises 100% of organic carbon resulting from renewable raw materials and consequently 1.2×10⁻¹⁰% by weight of ¹⁴C, with regard to the total weight of carbon.

The ¹⁴C content of the methyl methacrylate can be measured, for example, according to the following techniques:
by liquid scintillation spectrometry: this method consists in counting the “β” particles resulting from the disintegration of the ¹⁴C. The β radiation resulting from a sample of known weight (known number of carbon atoms) is measured for a certain time. This “radioactivity” is proportional to the number of ¹⁴C atoms, which can thus be determined, The ¹⁴C present in the sample emits β⁻ radiation which, on contact with the liquid scintillant (scintillator), gives rise to photons. These photons have different energies (of between 0 and 156 keV) and form what is referred to as a ¹⁴C spectrum. According to two variants of this method, the analysis relates either to the CO₂ produced beforehand by combustion of the carbon-comprising sample in an appropriate absorbing solution or to the benzene after prior conversion of the carbon-comprising sample to benzene.
by mass spectrometry: the sample is reduced to graphite or to CO₂ gas and analyzed in a mass spectrometer. This technique uses an accelerator and a mass spectrometer in order to separate the ¹⁴C ions from the ¹²C ions and thus to determine the ratio of the two isotopes.

These methods for measuring the ¹⁴C content of the materials are specifically described in the standard ASTM D6866 (in particular D6866-06) and in the standard ASTM D7026 (in particular 7026-04). These methods compare the data measured on the analyzed sample with the data of a reference sample of 100% renewable origin, to give a relative percentage of carbon of renewable origin in the sample.

The measurement method preferably used in the case of methyl methacrylate is the mass spectrometry described in the standard ASTM D6866-06.

The following examples illustrate the present invention without, however, limiting the scope thereof. In these examples, the parts and percentages are by weight, unless otherwise indicated.

### EXAMPLE 1

### Manufacture of Syngas CO/H₂ and Isolation of the Carbon Monoxide

In the present example use is made of an ethanol/water mixture, the ethanol being obtained by ethanolic fermentation of sugar, as follows:

A water/sugar (10 kg of sugar) mixture is poured into a 50 liter plastic tank. 0.25 l of baker's yeast mixed beforehand with 0.25 l of tepid water, and a dose of Calgon (water softener) are added to the mixture and the combined product is allowed to soak at a temperature of 25° C. for 14 days. In order to limit the formation of acetic acid, the container is covered with a lid provided with a valve. On conclusion of this stage, the mixture is filtered and separated by settling, and the solution is distilled in order to recover the azeotrope of the ethanol, at 96% in water.

This ethanol/water mixture is subjected to a pressure of 30 bar and to a temperature of 900° C., with an Ni/alumina catalyst. At the outlet of the reactor, the excess water is condensed, along with the heavy impurities.

The CO/H₂ mixture is separated cryogenically, the mixture being passed into a liquid nitrogen trap in order to retain the CO. The condensed gas is subsequently reheated in order to separate the CO from the other impurities (methane, CO₂, etc.).

### EXAMPLE 2

### Manufacture of Methanol From Syngas

For the synthesis of methanol, use is made of syngas from example 1. The composition of this gas is adjusted in order to have an H₂/CO/CO₂ ratio of 71/23/6 and the CO₂ content is 6%. The total pressure of gas is 70 bar.

Use is made of a commercial Cu/Zn/Al/O catalyst MegaMax 700. The reactor is fed with the gas mixture at 70 bar with an HSV of 10 000 h⁻¹, which mixture passes over the catalyst at a temperature of 240° C. The mixture of the gases produced is subsequently reduced in pressure to atmospheric pressure and the methanol produced is isolated by distillation.

The selectivity for methanol is 99% and the methanol yield is 95%.

### EXAMPLE 3

### Manufacture of Isobutanol

The isobutanol may be isolated from a mixture known as fusel alcohols. In the present case, a commercially available mixture is used. This mixture contains 12.4 wt % ethanol, 3.5 wt % n-propanol, 9.5 wt % isobutanol and 74.6 wt % isoamyl alcohol. All the percentages being given without taking into account water. The mixture of fusel alcohols is obtained from an ethanol distillery. The mixture of fusel alcohols is firstly treated with an equivalent volume of hexane, and the water is removed by phase separation. After removing the water, sodium sulfate is added (around 0.15 kg of salt per liter of fusel alcohol) in order to reduce the water content in the fusel alcohol.

The alcohol mixture is then distilled to give various fractions. The fraction containing the isobutanol is isolated and the purity thereof is monitored by gas chromatography. The fraction rich in isobutanol also contains traces of ethanol (5 wt %) and of isoamyl alcohol (7 wt %). The mixture is then taken up again for a new distillation in order to have isobutanol containing less than 1% of each of the impurities.

### EXAMPLE 4

### Manufacture of Isobutene

The isobutanol obtained in example 3 is evaporated with steam in order to create an equimolar mixture of isobutanol and water.

In a plant, the isobutanol is vaporized in a vaporizer, then preheated in a heat exchanger, before being injected at the top of a reactor with a diameter of 127 mm containing a catalytic bed brought to 300-400° C. and consisting of a layer of ESM110 alumina from Eurosupport, representing a volume of 12 700 cm³ and a weight of 6500 g, the ratio of the flow rate by volume of isobutanol to the volume of catalyst being 1 h⁻¹. The mixture of water and isobutene produced in the reactor is cooled in the heat exchanger, before being sent to a gas-liquid separator where the isobutene and the water (possibly mixed with by-products) are separated.

### EXAMPLE 5

### Manufacture of Methacrolein From Isobutene

The isobutene obtained in example 4 is used.

A reactor with a diameter of 2.54 cm and a length of 1 m, immersed in a molten salt bath at a temperature of 339° C. is fed at an HSV of 1000 h⁻¹ with a 2/1/2.5/12 O₂/isobutene/H₂O/N₂ mixture. The reactor is charged with YS79-1 catalyst from Nippon Kayaku. The hot spot in the catalyst bed reaches 412° C.

After operating for 300 hours, the conversion is 99%, the methacrolein yield is 79%, and the methacrylic acid yield is 4.0%.

### EXAMPLE 6

### Manufacture of Methacrylic Acid From Isobutene

Two reactors in series with diameters of 2.54 cm and lengths of 1 m, immersed in molten salt baths at temperatures of 367° C. and 313° C. respectively are fed at an HSV of 1000 h⁻¹ with a 2/1/2.5/12 O₂/isobutene/H₂O/N₂ mixture. The first reactor is charged with YS79-1 catalyst from Nippon Kayaku, and the second with K80 catalyst from Nippon Kayaku. The hot spot in the second catalyst bed reaches 330° C.

After operating for 300 hours, the conversion is 99%, and the methacrylic acid yield is 37.5%, and the conversion of methacrolein between the first reactor and the second reactor is 52%.

### EXAMPLE 7

### Manufacture of Methyl Methacrylate From Methacrylic Acid

For this step, the methacrylic acid obtained in the preceding step and the methanol obtained according to example 2 are used. The acid is brought into contact in the presence of a stabilizer (800 ppm of EMHQ) with a methacrylic acid/methanol ratio of 5, in a column fed from the bottom to the top containing a K2431 resin from Lanxess, maintained at 85° C. with a residence time of 70 minutes.

The product is collected and analyzed. After operating continuously for 15 hours, the product contains 75% of methacrylic acid and 18% of methyl methacrylate, which is recovered.

1. A process for the manufacture of methyl methacrylate comprising the steps of oxidation of methacrolein to methacrylic acid and esterification of said methacrylic acid with methanol, wherein at least one fraction of at least one from among methacrolein and methanol in this reaction was obtained by a reaction or a sequence of reactions starting from biomass.

2. The process as claimed in claim 1, wherein said oxidation and said esterification are carried out in two successive steps.

3. The process as claimed in claim 1, wherein said oxidation and said esterification are carried out simultaneously.

4. The process as claimed in claim 1, wherein at least one fraction of methanol was is obtained by pyrolysis of wood or by gasification of any material of animal and/or plant origin resulting in a syngas essentially composed of carbon monoxide and hydrogen, or by fermentation starting from plant crops, such as wheat, sugar cane or beet, giving fermentable products and thus alcohol,
it also being possible for at least one fraction of syngas for preparing the methanol to originate from the recovery of spent liquor and bleaching liquor from the manufacture of cellulose pulps.

5. The process as claimed in claim 1, wherein at least one fraction of methacrolein is obtained by oxidation of at least one from among isobutene, tert-butanol and/or a mixture of the two, the isobutene, optionally as a mixture with tert-butanol, resulting from the dehydration of isobutanol, it being possible for at least one fraction of isobutanol to have been obtained by distillation of a fusel oil, and/or by fermentation, in the presence of at least one yeast, of at least one plant material, which is generally in a hydrolyzed form before the fermentation, the fermentation being followed by a distillation step in order to recover the isobutanol in the form of an aqueous solution, which solution is subsequently subjected to a concentration step, and/or by condensation of methanol with ethanol, the methanol and/or the ethanol being derived from biomass.

6. The process as claimed in claim 1, wherein at least one fraction of methacrolein is obtained by oxidizing dehydrogenation of isobutyraldehyde, at least one fraction of this resulting from the reaction of propylene with a syngas and/or from the oxidation of isobutanol,
it being possible for at least one fraction of isobutanol to have been obtained by distillation of a fusel oil, and/or by fermentation, in the presence of at least one yeast, of at least one plant material, which is generally in a hydrolyzed form before the fermentation, the fermentation being followed by a distillation step in order to recover the isobutanol in the form of an aqueous solution, which solution is subsequently subjected to a concentration step, and/or by condensation of methanol with ethanol, the methanol and/or the ethanol being derived from biomass;
it being possible for at least one fraction of syngas to originate from the gasification of any material of animal or plant origin and/or from the recovery of spent liquor and bleaching liquor from the manufacture of cellulose pulps.

7. The process as claimed in claim 6, wherein at least one fraction of propylene was obtained by dehydration of isopropanol, said isopropanol obtained by biomass fermentation, or by metathesis reaction of ethylene and but-2-ene, obtained by dehydration of a mixture of alcohols, comprising at least ethanol and butan-1-ol, resulting from biomass fermentation.

8. The process as claimed in claim 1, wherein at least one fraction of methacrolein is obtained by reaction of propanaldehyde over formaldehyde,
at least one fraction of propanaldehyde possibly resulting from the hydrogenation of acrolein, at least one fraction of the latter originating from the dehydration of glycerol, at least one fraction of the latter having been able to be obtained as a by-product of the manufacture of biofuels starting from oleaginous plants, comprising triglycerides, a hydrolysis or a transesterification of these triglycerides making it possible to form glycerol in addition to fatty acids and fatty esters respectively; and
at least one fraction of formaldehyde by oxidation of methanol, at least one fraction of the methanol used having been obtained by pyrolysis of wood or by gasification of any material of animal or plant origin resulting in a syngas essentially composed of carbon monoxide and hydrogen, or by fermentation starting from plant crops, such as wheat, corn, sugar cane or beet, giving fermentable products and thus alcohol.

9. (canceled)

10. Methyl methacrylate produced by the process of claim 1, containing at least 0.4×10⁻¹⁰% by weight of ¹⁴C with regard to the total weight of carbon.

11. The process of claim 1 further comprising one of the following steps:
a) polymerizing said methyl methacrylate to form a poly(methyl methacrylate) polymer or copolymer;
b) reacting said methyl methacrylate by organic synthesis to form higher methacrylates;
c) polymerizing said methyl methacrylate to form acrylic emulsions, acrylic resins, additives for poly(vinyl chloride), and as an additive for lubricants.
